(19) United States
(12) Patent Application Publication
(10) Pub. No.: US 2010/0143435 A1
(43) Pub. Date: Jun. 10, 2010

(54) SCAFFOLD WITH INCREASED PORE SIZE

(75) Inventors: Anthony Dagger, Heslington (GB); Helene Lecomte, Heslingtonde (GB); Rhianna Moss, Heslington (GB)

Correspondence Address: FISH & RICHARDSON P.C.;Smith & Nephew, Inc., 1450 Brooks Road, Memphis, TN, 38116, US

(73) Assignee: SMITH & NEPHEW PLC, London (GB)

(21) Appl. No.: 12/525,723

(22) PCT Filed: Feb. 8, 2008
(86) PCT No.: PCT/GB08/00438

§ 371 Date: Feb. 1, 2010

(30) Foreign Application Priority Data
Feb. 14, 2007 (GB) 0702847.5

(51) Int. Cl. A61K 9/00; B29C 47/00; C12N 5/071; A61K 45/00; A61P 43/00

(52) U.S. Cl. 424/422; 264/465; 435/395; 435/396; 424/93.7

(57) ABSTRACT

The invention relates to scaffolds for use as medical devices, for guided tissue regeneration and repair, wherein the relationship between fibre diameter and pore size in a scaffold is decoupled, thereby enabling the small fibre diameters required for cell attachment and proliferation and the large pore sizes needed for cell migration into the scaffold to be achieved.

### FIELD OF THE INVENTION

The present invention relates to scaffolds which can be used as medical devices for guided tissue regeneration and repair.

### BACKGROUND TO THE INVENTION

Electrospinning is a commonly used polymer processing technique used to generate fibrous scaffolds and membranes having a wide range of fibre diameter and pore dimensions. These scaffolds are typically in the form of a non-woven fabric, resulting from the random deposition of the polymer fibres onto a target. In medical applications such scaffolds encourage the in-growth of host cells, which in turn deposit a natural extracellular matrix as the biodegradable polymer(s) of the scaffold degrade.

A well documented relationship exists between the diameter of the electrospun polymer fibres and the pores between these fibres. Lower concentration polymer solutions lead to the formation of small fibres and small individual pore size (Boland et al., 2004). As the polymer concentration increases, both the fibre diameters and pore sizes increase and the cellular response to these changes.

Whilst the presence of thin fibres in electrospun scaffolds provides a larger surface area for cell attachment and proliferation, the downside is that these fibres are associated with small pores sizes, which are detrimental to cell migration through the scaffold (Lannutti et al., 2006).

In order to maximise all three of these cell behaviours, the ideal scaffold structure contains thin fibres which provide an optimal surface area to promote cell attachment and proliferation and an optimal pore size to promote cell migration. To achieve this requires the decoupling of the relationship between fibre diameter and pore size.

Various methods have been employed to create an increased pore size in standard scaffolds, including: particle-leaching using porogens, foaming using blowing agents, ablation using a laser, an electron beam or mechanical perforation, and the use of electrospun fibres having different chemical properties.

Electrospun composite scaffolds in which increased porosity is created by one type of polymer fibre being removed in-situ following implantation of the scaffold, as a result of a higher rate of degradation in comparison to the other polymer(s), are known (Pham et al., 2006). Although this ultimately creates larger pores relative to the pore size in the scaffold prior to implantation, this optimal pore geometry is not available from the very beginning of use of the device, which will subsequently delay the cellular response (Lannutti et al., 2006).

There is therefore a need for a scaffold having optimal architecture at the time of implantation.

### STATEMENTS OF THE INVENTION

This invention enables the relationship between fibre diameter and pore size in a scaffold to be decoupled, thereby enabling the small fibre diameters required for cell attachment and proliferation and the large pore sizes needed for cell migration into the scaffold to be achieved.

Thus according to an aspect of the invention there is provided a method of manufacturing a polymer scaffold, the method comprising the steps of;
(a) generating a first set of fibres from a first polymer solution;
(b) generating a second set of fibres from a second polymer solution, wherein the second set of fibres are interspersed between the first set of fibres; and,
(c) extracting the second set of fibres from the scaffold.

The first set of fibres are those fibres which form the final scaffold. The second set of fibres are the “sacrificial” fibres, that is, those fibres which are removed from the scaffold prior to implantation in order to create the optimal pore size.

The concentrations of the polymer solutions are chosen such that they produce sets of fibres with different fibre diameters. The diameter of the second set of fibres being greater than the diameter of the first set of fibres, such that following removal of the second set of fibres, the pores formed are larger than if the first set of fibres had been generated in isolation.

In an embodiment of this invention the scaffold is generated by electospinning, wherein at least two polymer solutions are electrospun to form a non-woven, fibrous scaffold.

Thus in a further embodiment of the invention there is provided a method of manufacturing an electrospun polymer scaffold, the method comprising the steps of;
(a) dispensing within an electrostatic field in a direction of a target, a first polymer solution from a first dispenser, so as to form at least one jet of said first polymer solution;
(b) dispensing within an electrostatic field in a direction of the target, a second polymer solution from a second dispenser, so as to form at least one jet of said second polymer solution;
(c) collecting the at least one jet produced in each of steps (a) and (b) on the target to form a polymer scaffold;
(d) extracting the fibres formed from the second polymer solution from the scaffold.

In some embodiments of the invention the first and second polymer solutions are simultaneously dispensed onto the target. This results in a substantially homogeneous distribution of the first and second sets of polymer fibres throughout the scaffold.

In alternative embodiments of the invention the first and second polymer solutions are dispensed separately, for example pulsed. This can, for instance, result in a more localised or focused distribution of the first and second sets of polymer fibres within the scaffold.

The method may further comprise the step of drying the scaffold prior to extraction of the fibres formed from the second polymer solution, thereby minimising the amount of residual solvent retained within the scaffold prior to the extraction step.

In a further embodiment of the invention the scaffold is generated by thermal-induced phase separation (TIPS). In this process a variety of parameters such as type of polymer, polymer concentration, solvent/nonsolvent ratio, and quenching temperature influence the type of micro- and macroporous structures formed. For example, TIPS has been used to form tissue engineering scaffolds in which heat treatment causes polymer particles [for example poly(L-lactic acid)] to fuse and form a continuous fibrous matrix containing entrapped particles or porogens (for example NaCl) in a globular phase (Lee, 2004), This later phase is the sacrificial phase. The pores created by the removal of this globular phase are typically several hundred microns in diameter, which is some several fold larger than the diameter of the actual fibres. The ability to use TIPS to produce fibrous structures in both phases, particularly in the sacrificial phase, allows significantly more control over the resulting pore size and also enables the generation of smaller pores. For example, the ability to generate pore sizes of between about 20-50 μm is advantageous as this more closely mimics the natural cellular environment.

The polymers used in the present invention can be natural, synthetic, biocompatible and/or biodegradable.

The term “natural polymer” refers to any polymers that are naturally occurring, for example, silk, collagen-based materials, chitosan, hyaluronic acid and alginate.

The term “synthetic polymer” means any polymers that are not found in nature, even if the polymers are made from naturally occurring biomaterials. Examples include, but are not limited to aliphatic polyesters, poly(amino acids), copoly(etheresters), polyalkylenes, oxalates, polyamids, tyrosine derived polycarbonates, poly(iminocarbonates), polyorthoesters, polyoxaesters, polyamidoesters, polyoxaesters containing amino groups, poly(anhydrides), polyphosphazenes and combinations thereof.

Suitable synthetic polymers for use in the invention can also include biosynthetic polymers based on sequences found in collagen, elastin, thrombin, fibronectin, starches, poly(amino acid), poly(propylene fumarate), gelatin, alginate, pectin, fibrin, oxidised cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, polynucleotides and combinations thereof.

In embodiments of the invention the biosynthetic polymers can be functionalised or modified variants of a natural polymer, for example, carboxymethylcellulose.

The term “biocompatible polymer” refers to any polymer which when in contact with the cells, tissues or body fluid of an organism does not induce adverse effects such as immunological reactions and/or rejections and the like.

The term “biodegradable polymer” refers to any polymer which can be degraded in the physiological environment such as by proteases. Examples of biodegradable polymers include, collagen, fibrin, hyaluronic acid, polylactic acid (PLA), polyglycolic acid (PGA), polycaprolactone (PCL), polydioxanone (PDO), trimethylene carbonate (TMC), polyethyleneglycol (PEG), alginate, chitosan or mixtures thereof.

In an embodiment of the invention the first polymer solution comprises at least one biocompatible polymer and/or biodegradable polymer. In particular embodiments of the invention the first polymer solution comprises a glycolide, and specifically comprises over 85% glycolide, over 90% glycolide, over 95% glycolide, or consists of 100% glycolide.

Polyglycolic acid (PGA), also referred to as polyglycolide, is a biodegradable, thermoplastic polymer and the simplest linear, aliphatic polyester. It can be prepared starting from glycolic acid by means of polycondensation or ring-opening polymerisation of glycolide. PGA is characterised by hydrolytic instability owning to the presence of the ester linkage in its backbone and thus when it is exposed to physiological conditions, PGA is degraded by random hydrolysis. The degradation product, glycolic acid, is non-toxic and it can enter the tricarboxylic acid cycle after which it is excreted as water and carbon dioxide. The polymer has been shown to be completely resorbed by an organism in a time frame of four to six months.

In a particular embodiment of the invention the first polymer solution comprises PGA at a concentration of from about 5 to 15% w/w, particularly at a concentration of from about 5-10% w/w, and more particularly at a concentration of about 8% w/w.

In further embodiments of the invention the first polymer solution comprises copolymers or blends of (co)polymers. This can impart physical and/or chemical properties on the fibre formed which are in addition to those exhibited by a fibre formed from a single polymer.

Suitable examples of copolymers include copolymers of a glycolide and/or a lactide and/or other suitable hydroxy acids. Examples include poly(lactic-co-glycolic) acid (PLGA), a co-polymer with lactic acid; poly(glycolide-co-caprolactone) (PGACL), a co-polymer with ε-caprolactone and poly(glycolide-co-trimethylene carbonate) (PGATMC), a co-polymer with trimethylene carbonate.

In embodiments of the invention the copolymer is poly(lactide-co-glycolide) (PLGA), wherein the ratio of PGA:PLA is about 85:15, or about 85.25:14.75, or about 85.50:14.50, or about 85.75:14.25; or about 90:10, or about 90.25:9.75; or about 90.50:9.50; or about 90.75:9.25; or about 91:9; or about 92:8; or about 93:7; or about 94:6; or about 95:5; or about 96:4; or about 97:3; or about 98:2; or about 99:1.

The invention further covers blends of PGA and a polyester. Examples of suitable blends include polyglycolic acid blended with polylactic acid (PGA/PLA) and also polydioxanone blended with polyglycolic acid (PDO/PGA). It is envisaged that the blends can consist of at least one co-polymer.

All sterioisomeric forms of the polymers are envisaged.

The fibres formed from the first polymer solution advantageously have an average diameter of less than 10 μm, more particularly between about 10 nm and 10 μm, or between about 500 nm and 5 μm or between about 1 μm and 5 μm. Scaffolds comprising fibres having this diameter have been found to demonstrate an optimal architecture for cell attachment and proliferation.

It is not a requirement for the polymer(s) of the second polymer solution to be biocompatible and/or biodegradable as these are the sacrificial fibres which are extracted prior to implantation of the scaffold into the body. Any polymer that, for example, dissolves in a solvent that the first polymer does not dissolve in, or melts at a significantly lower temperature than the first polymer, or is degraded by an enzyme that doesn't degrade the first polymer, in order that the fibres can be removed without altering the structure of the first polymer, is appropriate.

Suitable solvents include, but are not limited to, halogenated solvents, such as chlorinated or fluorinated solvents, aqueous solutions or ionic liquids

In an embodiment of the invention the second polymer solution comprises polycaprolactone (PCL) at a concentration of about 10-20% w/w, and more particularly at a concentration of about 15% w/w.

As an example of this embodiment of the invention the, first set of polymer fibres comprise PGA and the second set of polymer fibres comprises PCL. PGA has a melting point of about 225-230° C. whereas PCL has a melting point of about 58-63° C. This distinct difference in melting points of the two sets of fibres can be exploited in order to remove the second set of fibres whilst retaining the first set of fibres intact.

Following the extraction of the fibres formed from the second polymer solution, the scaffold advantageously has an average pore dimension of between about 10-20 μm, and more advantageously about 15 μm. Scaffolds having this pore size have been found to demonstrate an optimal architecture for cell migration.

It is further envisaged that the first set of fibres are porous thereby allowing migration of cells and the penetration of oxygen and nutrients throughout the fibres. The pores can be on the micro- or nano-scale.

The pores can be achieved during fibre formation by varying, for example, the electrospinning conditions as would be known to those skilled in the art.

Alternatively the porosity can be achieved post-fibre formation. For example, by the mechanical perforation of the fibre. Alternatively the first set of fibres upon formation can comprise a co-polymer, blend of polymers, or pore generating additives (porogens) with these components being extracted from the fibre post-formation, resulting in a porous fibre. The extraction step can be based on, for example, solvent dissolution or temperature differences.

In an embodiment of the invention it is envisaged that the first set of fibres comprises a blend of polymer X and polymer Y, whilst the second set of fibres consists of polymer Y. Extraction of polymer Y from the scaffold, results in (i) a porous architecture between the first set of fibres and (ii) porosity within the first set of fibres.

For example, the first set of fibres comprises a blend of PGA and PCL (PGA/PCL), whilst the second set of fibres consists of PCL. Solvent extraction, using for example, dichloromethane of the PCL results in the removal of the second set of fibres and also a perforated first set of fibres.

Whilst the extractable polymer within the first set of first fibres and the second set of fibres can be the same polymer, in further embodiments of the invention, the extractable polymers can be different polymers. For instance, the first set of fibres comprises a blend of polymer X and polymer Y, whilst the second set of fibres consists of polymer Z. Extraction of polymer Y from the scaffold results in a porous first set of fibres, whilst extraction of polymer Z from the scaffold results in large pores disposed between the first set of fibres.

In order to increase the bioaffinity and recognition of the cells proliferating and/or migrating through the electrospun scaffold and/or to increase the therapeutic potential of the scaffold, it is envisaged that at least one agent for promoting cell colonisation, differentiation, extravasation and/or migration is associated with fibre formed from the first polymer solution. This at least one agent can be a biological, chemical or mineral agent, which can be attached to, embedded within or impregnated within this fibre. The agent can be provided within the first polymer solution such that during electrospinning the agent becomes associated with the fibre. Additionally or alternatively the at least one agent can be associated with the fibre post-electrospinning.

The scaffold can comprise cells, which can be associated with the scaffold, either during or after fibre formation. Examples of appropriate cells include fibroblasts, epidermal cells, dermal cells, epithelial cells and keratinocytes.

According to a second aspect of the invention there is provided a scaffold manufactured according to the first aspect of the invention.

According to a further aspect of the invention there is provided a medical dressing comprising or consisting of the scaffold manufactured according to the first aspect of the invention.

In an embodiment of this aspect of the invention, the medical dressing is a wound dressing.

According to a further aspect of the invention there is provided a method of inducing ex vivo formation of a tissue, the method comprising the steps of:
(a) providing a scaffold as manufactured according to the present invention;
(b) seeding the scaffold with cells in a medium selected suitable for proliferation, differentiation, and/or migration of said cells to thereby induce the formation of the tissue.

According to a further aspect of the invention there is provided a method of inducing in vivo formation of a tissue in a subject the method comprising the steps of:
(a) providing a scaffold as manufactured according to the present invention;
(b) implanting the scaffold into the subject to thereby induce the formation of the tissue.

In embodiments of the invention the scaffold is implanted into a dermal wound bed to promote tissue formation at a wound site.

According to a still further aspect of the invention there is provided a method of treating a subject having a pathology characterised by a tissue damage or loss, the method comprising the steps of;
(a) providing a scaffold as manufactured according to the present invention.
(b) implanting the scaffold into the subject to thereby induce the formation of the tissue and treat the subject.

### DETAILED DESCRIPTION OF THE INVENTION

Method

Solution Preparation

Solutions of polyglycolic acid (PGA, Mw=116,000 g.mol-1) at 8% w/w, and polycaprolactone (PCL, 37,000 g.mol-1) at 15% w/w are prepared in hexafluoroisopropanol (HFIP).

Electrospinninq

The two polymer solutions are loaded into separate 10 ml syringes and placed into a syringe pump set to dispense the solutions at 0.03 ml/minute. Flexible plastic tubing (internal diameter 1.5 mm) is used to connect the syringe exits to metallic 18-gauge needles, which are filed down to remove the taper. One needle is clamped vertically above the target with a working distance (from needle tip to target) of 15 cm, the other horizontally in front of the target with a working distance of 10 cm. Both needles are connected to the live port of a high-voltage generator.

The target is a cylindrical aluminium mandrel (5 cm diameter×10 cm long) attached to a motor. The motor enables the target to be rotated at 50 rpm to collect an even layer of nanofibrous material. The target is earthed, and covered in replaceable baking paper to ease the release of the formed nanofibrous material.

The electrospinning process is initiated by applying a voltage of ˜10 kV to the needles using a Glassman voltage generator, while the target is earthed. Electrospinning begins when the voltage applied to the needles is sufficient enough to prevent the polymer solutions from dripping and allows them to be drawn towards the rotating target as jets, these polymer jets are then collected on the baking paper as a mixture of two sets of fibres. The minimum voltage required to initiate the electrospinning process is normally used and the amount of time the process runs for is dependant upon the depth of scaffold required.

The scaffolds produced are vacuum dried to minimise the amount of residual solvent.

Rinsing Step

After drying, the scaffolds containing the two sets of polymer fibres are rinsed in dichloromethane (DCM) to remove all of the PCL fibres. The rinsing step is carried out by individually immersing the scaffolds in a beaker containing DCM (approximately 200 ml) for 5 minutes. This step is repeated as many times as necessary to ensure complete removal of the PCL fibres, as observed by DSC or any other suitable analytical method.

After rinsing, the scaffolds are once again vacuum dried to remove any trace of solvent.

Determination of Fibre Diameter and Pore Size

The electrospun scaffolds are imaged using a Scanning Electron Microscope (SEM), both before and after rinsing.

Fibre diameters and pore sizes are determined from the SEM images obtained. Measurements are performed either manually using a ruler and the scale bar or by using Image ProPlus software. For each sample, 30 fibres and 30 pores are randomly measured per SEM image and the mean and standard deviation of these are calculated.

Pore size is defined as the longest dimension per pore (usually a diagonal) and pores are defined as polygons created by intersecting fibres.

Results

Dimensions Obtained

Dimensions were measured for two types of electrospun mats

TABLE 1
Comparison of dimensions for standard and combination
electrospun structures

| Type of | Fibre diameter | Pore dimension |
|---|---|---|
| electrospun mat | (μm) | (μm) |
| Standard^{a} | 1.3 ± 0.5 | 8.6 ± 2.6 |
| Combination^{b} | 1.5 ± 0.4 | 14.5 ± 3.8 |

^{a}PGA electrospun in a standard fashion
^{b}PGA electrospun in combination with PCL, and PCL subsequently rinsed out

### REFERENCES

Boland et al., (2004). Utilizing acid pre-treatment and electrospinning to improve biocompatibility of PLGA for tissue engineering. J Biomed Mater Res. 15; 71B(1):144-152

Lannutti et al., (2006). Electrospinning for tissue engineering scaffolds. Materials Science and Engineering. Article in Press.

Pham et al., (2006). Electrospinning of polymeric nanofibers for tissue engineering applications: A Review. Tissue Engineering 12: 1197-1211.

Lee S H, Kim B S, Kim S H, Kang S W, Kim Y H. Thermally produced biodegradable scaffolds for cartilage tissue engineering 1: Macromol Biosci. 2004 Aug. 9;4(8):802-10.

1. A method of manufacturing a polymer scaffold, the method comprising the steps of:
(a) generating a first set of fibres from a first polymer solution;
(b) generating a second set of fibres from a second polymer solution, wherein the second set of fibres are interspersed between the first set of fibres; and,
(c) extracting the second set of fibres from the scaffold.

2. A method according to claim 1, wherein the scaffold is electrospun and the method comprising the steps of:
(a) dispensing within an electrostatic field in a direction of a target, a first polymer solution from a first dispenser, so as to form at least one jet of said first polymer solution,
(b) dispensing within an electrostatic field in a direction of the target, a second polymer solution from a second dispenser, so as to form at least one jet of said second polymer solution,
(c) collecting the at least one jet produced in each of steps (a) and (b) onto the target to form a polymer scaffold; and
(d) extracting the fibres formed from the second polymer solution from the scaffold.

3. A method according to claim 2, wherein the first and second polymer solutions are simultaneously dispensed onto the target.

4. A method according to claim 1, wherein the method further comprises the step of drying the scaffold prior to the extraction step.

5. A method according to claim 1, wherein the first and second sets of fibres are generated by thermal induced phase separation.

6. A method according to claim 1, wherein the first polymer solution comprises a biocompatible polymer.

7. A method according to claim 1, wherein the first polymer solution comprises a bioresorbable polymer.

8. A method according to claim 1, wherein the first polymer solution comprises a glycolide.

9. A method according to claim 8, wherein the glycolide comprises polyglycolic acid (PGA).

10. A method according to claim 1, wherein the second polymer solution comprises polycaprolactone (PCL).

11. A method according to claim 1, wherein the fibres formed from the second polymer solution are extracted using a solvent.

12. A method according to claim 11, wherein the solvent comprises a halogenated solvent.

13. A method according to claim 12, wherein the halogenated solvent is one of a chlorinated or fluorinated solvent.

14. A method according to claim 11, wherein the solvent is aqueous.

15. A method according to claim 11, wherein the solvent comprises an ionic liquid.

16. A method according to claim 1, wherein the fibres formed from the second polymer solution are extracted based upon differences in melting temperatures between the first and second sets of polymeric fibres.

17. A method according to claim 1, wherein the fibres formed from the second polymer solution are extracted via enzymatic degradation.

18. A method according to claim 1, wherein the fibres formed from the first polymer solution have a mean diameter of less than 10 μm.

19. A method according to claim 18, wherein the mean fibre diameter is between about 10 nm and 10 μm.

20. A method according to claim 19, wherein the mean fibre diameter is between about 500 nm and 5 μm.

21. A method according to claim 20, wherein the mean fibre diameter is between about 1 μm and 5 μm.

22. A method according to claim 1, wherein the scaffold, after extraction of the fibres formed from the second polymer solution, has an average pore dimension of between about 10-20 μm.

23. A method according to claim 22, wherein the average pore dimension is about 15 μm.

24. A method according to claim 1, further comprising at least one agent for promoting one or more of cell colonisation, differentiation, extravasation and migration associated with the polymer fibre formed from the first polymer solution.

25. A method according to claim 24, wherein the agent is at least one of attached to, embedded within and impregnated within the polymer fibre formed from the first polymer solution.

26. A method according to claim 24, wherein the agent is associated during fibre formation.

27. A method according to claim 24, wherein the agent is associated after post fibre formation.

28. A method according to claim 1, wherein the method further comprises incorporating the scaffold into a wound dressing.

29. A method of inducing ex vivo formation of a tissue, the method comprising:
(a) providing a scaffold as manufactured according to claim 1;
(b) seeding the scaffold with cells in a medium selected suitable for one or more of proliferation, differentiation, and migration of said cells to thereby induce the formation of the tissue.

30. A method of inducing in vivo formation of a tissue in a subject, the method comprising:
(a) providing a scaffold as manufactured according to claim 1;
(b) implanting the scaffold into the subject to thereby induce the formation of the tissue.

31. A method according to claim 30, wherein the scaffold is implanted into a dermal wound bed.

32. A method of treating a subject having a pathology characterised by a tissue damage or loss, the method comprising:
(a) providing a scaffold as manufactured according to claim 1;
(b) implanting the scaffold into the subject to thereby induce the formation of the tissue and treat the subject.

33. A medical dressing manufactured according to the method of claim 1.
